(19) United States
(12) Patent Application Publication
(10) Pub. No.: US 2010/0160699 A1
(43) Pub. Date: Jun. 24, 2010

(54) METHOD FOR EFFICIENT USE OF HYDROGEN IN AROMATICS PRODUCTION FROM HEAVY AROMATICS

(76) Inventors: Stanley Joseph FREY, Palatine, IL (US); Vasant P. Thakkar, Elk Grove Village, IL (US)

Correspondence Address: HONEYWELL/UOP;PATENT SERVICES, 101 COLUMBIA DRIVE, P O BOX 2245 MAIL STOP AB/2B, MORRISTOWN, NJ, 07962, US

(21) Appl. No.: 12/342,867

(22) Filed: Dec. 23, 2008

(51) Int. Cl. C07C 2/00; C10G 67/00

(52) U.S. Cl. 585/316; 208/97; 585/315

(57) ABSTRACT

A method for efficient use of hydrogen in aromatics production from heavy aromatic oil. A hydrocarbonaceous stream comprising C₉⁺ hydrocarbons and an essentially pure hydrogen stream are hydrotreated and hydrocracked to produce a hydrocracking zone effluent comprising aromatics. The hydrocracking zone effluent is fractionated to separately recover C₄ and lighter hydrocarbons, hydrocarbons boiling between about 180° F. and about 380° F., and diesel. The heavier hydrocarbons are combined with a low purity hydrogen-containing stream and heated, then dehydrogenated or transalkylated to form hydrogen, volatile compounds, and aromatics. The hydrogen and volatile components are separated from the aromatics and treated by pressure swing adsorption to provide an essentially pure hydrogen-containing stream, which is compressed and provided to the hydrotreating and hydrocracking steps. Liquid products are recovered from the aromatics-containing stream.

### FIELD OF THE INVENTION

The invention relates to a method for efficiently using hydrogen in aromatics production from heavy aromatic oil. In particular, the invention relates to a method for balancing hydrogen needs in a petroleum refinery by providing a once-through path for hydrogen by first routing a hydrogen stream through an aromatics reaction zone, then through purification and compression to be used in high-pressure hydrotreating and hydrocracking reactors.

### DESCRIPTION OF RELATED ART

In petrochemical refining, streams of various compositions are required as feedstocks to processing steps. These processing steps then typically produce by-product streams useful in other processing steps. For example, a pure, high pressure stream of hydrogen is required for high-pressure hydrotreating and hydrocracking. However, there seldom is such a stream produced as a by-product from another process. Therefore, purification of a stream that includes impurities deleterious to the hydrotreating and hydrocracking processes typically is required. Further, the hydrogen demand for such a unit is high, so a relatively large supply is required. The hydrogen stream also must be compressed to a relatively high pressure.

Similarly, a relatively small amount of hydrogen is required for formation of aromatics by transalkylation after dehydrogenation of a midcut (light naphtha) from fractionation of high-pressure hydrocracking reaction product. Typically, a hydrogen-containing stream from the transalkylation/cracking reactor is recycled to a dehydrogenation reactor typically immediately preceding the transalkylation/cracking reactor. These reactors require a high hydrogen/hydrocarbon ratio, but do not consume much hydrogen. Therefore, a compressor is necessary to move this recycle stream from one reactor to the other, and an impure hydrogen stream is a by-product.

Refiners therefore attempt to arrange stream flow paths to maximize use of such by-product streams. Often, it is not practical to use a single stream in a first and second process serially because the first process introduces compounds into the stream that are not suitable for the second process. Also, heat loads and compression requirements must be considered. Therefore, considerable effort is expended to develop flow paths that most efficiently use both by-product streams generated by processes and the feeds to these processes.

The compositions of these streams is important, to ensure not only that catalysts, resins, and other processing materials are not contaminated, but also that compounds are present in the reactors in proportions that enable the reaction to be carried out efficiently and effectively.

Many control and recycle systems are known in the prior art. For example, U.S. Pat. No. 3,974,064 is directed to a system for controlling a hydrogen/hydrocarbon ratio in catalytic reaction having a gas recycle.

U.S. Pat. No. 4,053,388 is directed to integration of a hydrotreater and a catalytic reformer in the manufacture of xylenes operated in hydrogen balance with each other.

U.S. Pat. No. 4,362,613 discloses a system in which a hydrocracking vapor is contacted with a permeable membrane to separate hydrogen from the vapor, compressing it, and returning it to the hydrocracking zone.

U.S. Pat. No. 4,929,794 is directed to a combination of hydrotreating and isomerization processes using a common source of hydrogen. The combination provides desired low hydrogen to hydrocarbon ratio in both processes.

U.S. Pat. No. 5,332,492 discloses recovery of hydrogen-rich gas by a particular arrangement of refrigeration, pressure swing adsorption, and separation processes that is particularly useful when integrated with hydrogen-consuming processes such as hydrocracking.

U.S. Pat. No. 6,179,900 is directed to a method for separating hydrogen from refinery off-gases such as catalytic cracker gas by selectively permeable membranes.

U.S. Pat. No. 6,280,609 discloses both heat exchange and catalyst movement to simplify equipment and minimize catalyst deactivation rate and therefore minimize regeneration requirements.

U.S. Pat. No. 7,252,702 is directed to a method of hydrogen recovery in petroleum refineries and petrochemical plants in which streams typically fed to plural pressure swing adsorption units are instead combined and fed to a single pressure swing adsorption unit. The method lowers the load on a steam reformer because the feed stream is reduced, and burning of excess as fuel gas or flared material is reduced.

U.S. Pat. No. 7,265,252 discloses selective hydrocracking of a feedstock, then introduction to a transalkylation zone with a stream high in benzene, toluene, and C₉⁺ hydrocarbons.

U.S. Pat. No. 7,268,263 is directed to recycle of selected aliphatic hydrocarbons to the isomerization unit of a xylene recovery zone to increase efficiency of the unit.

U.S. Pat. No. 7,271,303 discloses a multi-zone process for production of low sulfur diesel and aromatic compounds wherein C₉⁺ hydrocarbons are hydrocracked to produce low sulfur diesel and a naphtha-boiling stream that is reformed and transalkylated to produce a xylene-containing stream and to balance hydrogen needs. Hydrogen is compressed in a first stage, forwarded to the reforming/transalkylation zone. A hydrogen-rich stream then is recovered from this zone, pressurized in a second stage, and delivered to the hydrotreating/hydrocracking zone.

U.S. Pat. No. 7,288,687 discloses recycle of selected aliphatic hydrocarbons to the isomerization unit of a xylene recovery zone to increase efficiency of the unit.

As can be seen, there exists a need for an energy efficient method for efficiently using hydrogen in aromatics production from heavy aromatic oil that simplifies the processing scheme to reduce both operating and capital costs.

### SUMMARY OF THE INVENTION

In a first embodiment, the invention is directed to a method for efficiently using hydrogen in aromatics production from heavy aromatic oil that is energy efficient and which simplifies the processing scheme to reduce both operating and capital costs

In a second embodiment, the invention is directed to a method for balancing hydrogen needs in a petroleum refinery by providing a once-through path for hydrogen by first routing a hydrogen stream through an aromatics reaction zone, then through purification and compression to be used in high-pressure hydrotreating and hydrocracking reactors.

Another embodiment of the invention is directed to reducing or eliminating the need for a gas recycle in the aromatic production zone.

### BRIEF DESCRIPTION OF THE DRAWINGS

FIG. 1 shows a schematic flow chart of an embodiment of the invention.

FIG. 2 illustrates a schematic flow chart for an embodiment of the invention.

### DETAILED DESCRIPTION OF THE INVENTION

Petroleum refining is complex and requires plural processing units. Products from one unit typically may be delivered to other units for additional processing. To increase operating efficiency, by-product streams from units often are used in other units as feedstocks. Embodiments of this invention are directed to improving process flow, minimizing capital investment, and improving processing efficiency in the production of diesel and aromatics. In particular, embodiments of this invention are directed to the portion of a petroleum refinery including C₉⁺ hydrotreating and hydrocracking units, through and including xylenes and other products.

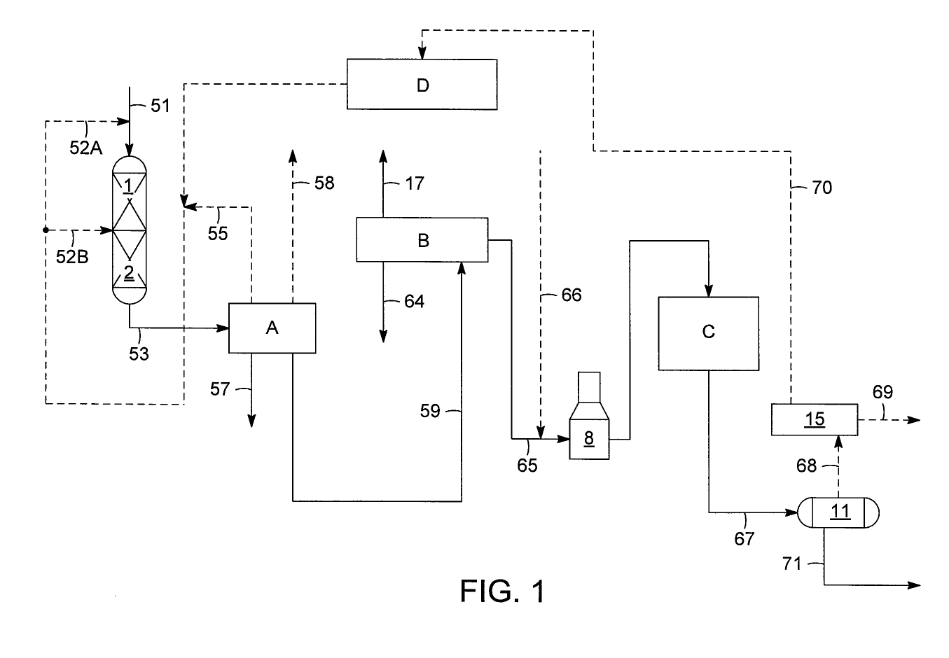

An embodiment of the invention is illustrated in FIG. 1. Heavy oil aromatic-rich feed 51 is introduced to hydrotreating reactor 1, and then to hydrocracking reactor 2. High-purity hydrogen is introduced into each reactor separately at 52A and 52B. Hydrotreating reactor 1 is a denitrification and desulfurization reactor. Preferred denitrification and desulfurization reaction conditions or hydrotreating reaction conditions include a temperature from about 204° C. (400° F.) to about 482° C. (900° F.), a pressure from about 3.5 MPa (500 psig) to about 17.3 MPa (2500 psig), a liquid hourly space velocity of the fresh hydrocarbonaceous feedstock from about 0.1 to about 10 hr⁻¹ with a hydrotreating catalyst or a combination of hydrotreating catalysts.

The term “hydrotreating” as used herein refers to processes wherein a hydrogen-containing treat gas is used in the presence of suitable catalysts which are primarily active for the removal of heteroatoms, such as sulfur and nitrogen. Suitable hydrotreating catalysts for use in the present invention are any known conventional hydrotreating catalyst and include those which are comprised of at least one Group VIII metal, preferably iron, cobalt and nickel, more preferably cobalt and/or nickel and at least one Group VI metal, preferably molybdenum and tungsten, on a high surface area support material, preferably alumina. Other suitable hydrotreating catalysts include zeolitic catalysts, as well as noble metal catalysts where the noble metal is selected from palladium and platinum. It is within the scope of the present invention that more than one type of hydrotreating catalyst be used in the same reaction vessel. The Group VIII metal is typically present in an amount ranging from about 2 to about 20 wt-percent, preferably from about 4 to about 12 wt-percent. The Group VI metal will typically be present in an amount ranging from about 1 to about 25 wt-percent, preferably from about 2 to about 25 wt-percent. Typical hydrotreating temperatures range from about 204° C. (400° F.) to about 482° C. (900° F.) with pressures from about 3.5 MPa (500 psig) to about 17.3 MPa (2500 psig), preferably from about 3.5 MPa (500 psig) to about 13.9 MPa (2000 psig).

In accordance with a preferred embodiment of the present invention, the resulting effluent from the denitrification and desulfurization zone is introduced into a hydrocracking zone 2 together with hydrogen 52B. The hydrocracking zone may contain one or more beds of the same or different catalyst. In one embodiment the preferred hydrocracking catalysts utilize amorphous bases or low-level zeolite bases combined with one or more Group VIII or Group VIB metal hydrogenation components. In another embodiment, the hydrocracking zone contains a catalyst which comprises, in general, any crystalline zeolite cracking base upon which is deposited a minor proportion of a Group VIII metal hydrogenating component. Additional hydrogenation components may be selected from Group VIB for incorporation with the zeolite base. The zeolite cracking bases are sometimes referred to in the art as molecular sieves and are usually composed of silica, alumina and one or more exchangeable cations such as sodium, magnesium, calcium, rare earth metals, etc. They are further characterized by crystal pores of relatively uniform diameter between about 4 and 14 angstroms. It is preferred to employ zeolites having a silica/alumina mole ratio between about 3 and 12. Suitable zeolites found in nature include, for example, mordenite, stillbite, heulandite, ferrierite, dachiardite, chabazite, erionite, and faujasite. Suitable synthetic zeolites include, for example, the B, X, Y, and L crystal types, e.g., synthetic faujasite and mordenite. The preferred zeolites are those having crystal pore diameters between about 8 and 12 angstroms, wherein the silica/alumina mole ratio is about 4 to 6. A prime example of a zeolite falling in the preferred group is synthetic Y molecular sieve.

The natural occurring zeolites are normally found in a sodium form, an alkaline earth metal form, or mixed forms. The synthetic zeolites are nearly always prepared first in the sodium form. In any case, for use as a cracking base it is preferred that most or all of the original zeolitic monovalent metals be ion-exchanged with a polyvalent metal and/or with an ammonium salt followed by heating to decompose the ammonium ions associated with the zeolite, leaving in their place hydrogen ions and/or exchange sites which have actually been decationized by further removal of water. Hydrogen or “decationized” Y zeolites of this nature are more particularly described in U.S. Pat. No. 3,130,006.

Mixed polyvalent metal-hydrogen zeolites may be prepared by ion-exchanging first with an ammonium salt, then partially back exchanging with a polyvalent metal salt and then calcining. In some cases, as in the case of synthetic mordenite, the hydrogen forms can be prepared by direct acid treatment of the alkali metal zeolites. The preferred cracking bases are those which are at least about 10 percent, and preferably at least 20 percent, metal-cation-deficient, based on the initial ion-exchange capacity. A specifically desirable and stable class of zeolites are those wherein at least about 20 percent of the ion exchange capacity is satisfied by hydrogen ions.

The active metals employed in the preferred hydrocracking catalysts of the present invention as hydrogenation components are those of Group VIII, i.e., iron, cobalt, nickel, ruthenium, rhodium, palladium, osmium, iridium and platinum. In addition to these metals, other promoters may also be employed in conjunction therewith, including the metals of Group VIB, e.g., molybdenum and tungsten. The amount of hydrogenating metal in the catalyst can vary within wide ranges. Broadly speaking, any amount between about 0.05 percent and 30 percent by weight may be used. In the case of the noble metals, it is normally preferred to use about 0.05 to about 2 wt-percent. The preferred method for incorporating the hydrogenating metal is to contact the zeolite base material with an aqueous solution of a suitable compound of the desired metal wherein the metal is present in a cationic form. Following addition of the selected hydrogenation metal or metals, the resulting catalyst powder is then filtered, dried, pelleted with added lubricants, binders or the like, if desired, and calcined in air at temperatures of e.g., 371° to 648° C. (700° to 1200° F.) in order to activate the catalyst and decompose ammonium ions. Alternatively, the zeolite component may first be pelleted, followed by the addition of the hydrogenating component and activation by calcining. The foregoing catalysts may be employed in undiluted form, or the powdered zeolite catalyst may be mixed and copelleted with other relatively less active catalysts, diluents or binders such as alumina, silica gel, silica-alumina cogels, activated clays and the like in proportions ranging between 5 and 90 wt-%. These diluents may be employed as such or they may contain a minor proportion of an added hydrogenating metal such as a Group VIB and/or Group VIII metal.

Additional metal promoted hydrocracking catalysts may also be utilized in the process of the present invention which comprises, for example, aluminophosphate molecular sieves, crystalline chromosilicates, and other crystalline silicates. Crystalline chromosilicates are more fully described in U.S. Pat. No. 4,363,718.

The hydrocracking of the hydrocarbonaceous feedstock in contact with a hydrocracking catalyst is conducted in the presence of hydrogen and preferably at hydrocracking reactor conditions which include a temperature from about 232° C. (450° F.) to about 468° C. (875° F.), a pressure from about 3.5 MPa (500 psig) to about 20.8 MPa (3000 psig), a liquid hourly space velocity (LHSV) from about 0.1 to about 30 hr⁻¹, and a hydrogen circulation rate from about 84 normal m³/m³ (500 standard cubic feet per barrel) to about 4200 m³/m³ (25,000 standard cubic feet per barrel). In accordance with the present invention, the hydrocracking conditions are selected on the basis of the feedstock with the objective of the production of aromatic compounds and diesel.

A hydrotreating/hydrocracking zone may contain one or more vessels or beds, each containing one or more types of hydrotreating catalyst or hydrocracking catalyst. Although liquid hydrocarbon recycle is not typical in embodiments of the invention, when a liquid hydrocarbon stream is recycled to the hydrotreating/hydrocracking zone, the recycle stream may be introduced directly into a hydrocracking catalyst, or may be passed through a bed of hydrotreating catalyst and then contacted with the hydrocracking catalyst.

In this embodiment, summarized in FIG. 1, the effluent from hydrocracking reactor is introduced into vapor/liquid separation zone A. In separation zone A, vapor streams are separated from liquid streams. Vapor streams can include recycle stream 55 and flash gas 58, the latter of which is removed from the system. Sour water stream 57 also is removed from the system, and hydrocarbon liquids 59 are removed for further processing.

Hydrocarbon liquids 59 then are processed in fractionation zone B to obtain various cuts of products in accordance with the operator's preference. Material streams that can be derived from fractionation zone B include LPG, light naphtha, diesel, and aromatics. The operator adjusts the operating conditions to obtain the preferred distribution of products.

As can be seen in FIG. 1, hydrocarbon liquid 59 is introduced to fractionation zone B. In fractionation zone B, diesel is separately recovered at 64, and LPG is separately recovered at stream 17.

Naphtha stream 65 is removed from fractionation zone A and combined with make-up hydrogen 66, as shown in FIG. 1. In an embodiment, naphtha stream 65 can include all hydrocarbons boiling above the boiling point of LPG and below that of diesel. Alternatively, in another embodiment, the lighter naphtha can be separately recovered by operation of additional fractionation in fractionation zone A.

Make-up hydrogen 66 typically is from a low-purity supply, such as gas from naphtha reforming or a hydrogen plant, and may include several contaminants, such as methane and other light hydrocarbons, and trace amounts of N₂, CO, CO₂, and H₂O.

The entirety of this heated stream is introduced to aromatic reaction zone C, which typically includes a dehydrogenator, a transalkylation reactor, or a combination of both reactors. Dehydrogenators also are known as reformers. The skilled practitioner recognizes that the dehydrogenation reaction is highly endothermic. Therefore, typically, plural reactors are used for this process, and the product flow is reheated to compensate for the net endothermic heat of reaction. However, in embodiments of the invention, such reheating is not necessary or is at least significantly reduced. In accordance with these embodiments of the invention, the relatively large quantity of the hydrogen make-up gas carries sufficient heat through the reactor(s). Thus, although plural reactors still may be used, there is no need, or reduced need, for inter-reactor heating in accordance with embodiments of the invention.

Any suitable reforming catalyst may be utilized if a reformer is present in aromatic reaction zone C. Preferred reforming catalysts contain a solid refractory oxide support having dispersed thereon at least one platinum group metal component and optionally a modifier metal component such as tin or rhenium. The support can be any of a number of well-known supports in the art including aluminas, silica/alumina, silica, titania, zirconia, and zeolites. The aluminas which can be used as support include gamma alumina, theta alumina, delta alumina, and alpha alumina with gamma and theta alumina being preferred. Included among the aluminas are aluminas which contain modifiers such as tin, zirconium, titanium, and phosphate. The zeolites which can be used include: faujasites, zeolite beta, L zeolite, ZSM 5, ZSM 8, ZSM 11, ZSM 12, and ZSM 35. The supports can be formed in any desired shape such as spheres, pills, cakes, extrudates, powders, granules, etc. and they may be utilized in any particular size.

One way of preparing a spherical alumina support is by the well known oil drop method which is described in U.S. Pat. No. 2,620,314. The oil drop method comprises forming an aluminum hydrosol by any of the techniques taught in the art and preferably by reacting aluminum metal with hydrochloric acid; combining the hydrosol with a suitable gelling agent; and dropping the resultant mixture into an oil bath maintained at elevated temperatures. The droplets of the mixture remain in the oil bath until they set and form hydrogel spheres. The spheres are then continuously withdrawn from the oil bath and typically subjected to specific aging and drying treatments in oil and ammoniacal solutions to further improve their physical characteristics. The resulting aged and gelled spheres are then washed and dried at a relatively low temperature of about 80° to 260° C. and then calcined at a temperature of about 455° to 705° C. for a period of about 1 to about 20 hours. This treatment effects conversion of the hydrogel to the corresponding crystalline gamma alumina. If theta alumina is desired then the hydrogel spheres are calcined at a temperature of about 950° to about 1100° C.

An alternative form of carrier material is a cylindrical extrudate, preferably prepared by mixing the alumina powder with water and suitable peptizing agents such as HCl until an extrudable dough is formed. The resulting dough is extruded through a suitably sized die to form extrudate particles. These particles are then dried at a temperature of about 260° to about 427° C. for a period of about 0.1 to 5 hours to form the extrudate particles. It is preferred that the refractory inorganic oxide comprises substantially pure alumina. A typical substantially pure alumina has been characterized in U.S. Pat. No. 3,852,190 and U.S. Pat. No. 4,012,313 as a by-product from a Ziegler higher alcohol synthesis reaction as described in Ziegler's U.S. Pat. No. 2,892,858.

An essential ingredient of the reforming catalyst is a dispersed platinum-group component. This platinum-group component may exist within the final catalytic composite as a compound such as an oxide, sulfide, halide, oxyhalide, etc., in chemical combination with one or more of the other ingredients of the composite or as an elemental metal. It is preferred that substantially all of this component is present in the elemental state and is uniformly dispersed within the support material. This component may be present in the final catalyst composite in any amount which is catalytically effective, but relatively small amounts are preferred. Of the platinum-group metals which can be dispersed on the desired support, preferred metals are rhodium, palladium, platinum, and platinum being most preferred.

A Group IVA (IUPAC 14) metal component is an optional ingredient of the reforming catalyst. Of the Group IVA (IUPAC 14) metals, germanium and tin are preferred and tin is especially preferred. This component may be present as an elemental metal, as a chemical compound such as the oxide, sulfide, halide, oxychloride, etc., or as a physical or chemical combination with the porous carrier material and/or other components of the catalytic composite. Preferably, a substantial portion of the Group IVA (IUPAC 14) metal exists in the finished catalyst in an oxidation state above that of the elemental metal.

Rhenium is also an optional metal promoter of the reforming catalyst. In addition to the catalytic components described above, other components may be added to the catalyst. For example, a modifier metal selected from the non-exclusive list of lead, indium, gallium, iridium, lanthanum, cerium, phosphorous, cobalt, nickel, iron, and mixtures thereof may be added to the reforming catalyst.

Another optional component of the reforming catalyst, particularly useful in embodiments of the present invention comprising dehydrogenation, dehydrocyclization, or hydrogenation reactions, is an alkali or alkaline-earth metal component. More precisely, this optional ingredient is selected from the group consisting of the compounds of the alkali metals—cesium, rubidium, potassium, sodium, and lithium—and the compounds of the alkaline earth metals—calcium, strontium, barium, and magnesium.

If a dehydrogenator is present, reactants may contact the catalyst in individual reactors in upflow, downflow, or radial flow fashion. The catalyst is contained in a fixed-bed system or in a moving-bed system with associated continuous catalyst regeneration. Alternative approaches to reactivation of deactivated catalyst are well known to those skilled in the art, and include semi-regenerative operation in which the entire unit is shut down for catalyst regeneration and reactivation or swing-reactor operation in which an individual reactor is isolated from the system, regenerated and reactivated while the other reactors remain on-stream.

Reforming conditions applied in the reforming zone in embodiments of the present invention typically include a pressure selected within the range of about 100 kPa (14.7 psig) to 7 MPa (1000 psig). Particularly good results are obtained at low pressure, namely a pressure of about 350 (50 psig) to 2750 kPa (400 psig). Reforming temperature is in the range from about 177° C. (350° F.) to about 565° C. (1049° F.). As is well known to those skilled in the reforming art, the initial selection of the temperature within this broad range is made primarily as a function of the desired product mix, sometimes measured in the form of octane, of the product reformate considering the characteristics of the charge stock and of the catalyst. Ordinarily, the temperature then is thereafter slowly increased during the run to compensate for the inevitable deactivation that occurs to provide a constant octane product.

Sufficient hydrogen is supplied by the make up hydrogen stream 66 to provide an amount of about 1 to about 20 moles of hydrogen per mole of hydrocarbon feed entering the reforming zone, with excellent results being obtained when about 2 to about 10 moles of hydrogen are used per mole of hydrocarbon feed. Likewise, the liquid hourly space velocity used in reforming is selected from the range of about 0.2 to about 20 hr⁻¹.

If a transalkylation reactor is present, the entire product stream from the dehydrogenator typically is introduced into the transalkylation reactor. Operating conditions preferably employed in the integrated reforming-transalkylation zone normally include a temperature from about 177° C. (350° F.) to about 550° C. (1022° F.) and a liquid hourly space velocity in the range from about 0.2 to about 10 hr⁻¹.

In accordance with an embodiment of the invention, use of this make-up hydrogen essentially obviates the need or purpose for a gaseous, hydrogen-rich recycle to balance the hydrogen needs of this aromatics zone. In some embodiments, a small recycle stream may still be present. The significant reduction in recycle results in a substantial savings in equipment, such as a recycle compressor and associate piping, and in operating costs, for example, for the operation of the recycle compressor. Further, these reactors typically are operated at pressures lower than the hydrotreating and hydrocracking reactors, so the make-up hydrogen stream 66 need not be compressed to the very high pressures used in the hydrotreating and hydrocracking reactors, thus resulting in significant savings in embodiments of the invention.

The resultant product 67 from aromatic reaction zone C then is introduced to vapor/liquid separator 11. Vapor/liquid separator 11 typically is operated at a gauge pressure between about 14 and about 30 bar. A liquid hydrocarbon in the naphtha boiling range is recovered as stream 71. This liquid hydrocarbon stream has greater molar aromatic flow than the naphtha. This stream can be further fractionated and products separately recovered or recycled. Hydrogen-rich gas stream 68 is purified in pressure-swing adsorption (PSA) unit 15. Tail gas stream 69 and purified hydrogen stream 70 are recovered from the PSA unit. In an embodiment, the hydrogen purity of stream 70 typically is at least about 95 percent, more typically at least about 97 percent, and most typically is at least about 98 percent.

Hydrogen stream 70 is compressed in high purity hydrogen compression zone D. The outlet pressure from zone D is sufficient to ensure that the hydrogen can be introduced into hydrotreating reactor 1 and hydrocracking reactor 2. High-purity, high pressure hydrogen from compression zone D can be mixed with an overhead gas stream 55 from vapor/liquid separation zone A and introduced separately into hydrotreating reactor 1 and hydrocracking reactor 2 at streams 52A and 52B, respectfully.

Thus, this once-through processing of hydrogen in accordance with embodiments of the invention produces a significant saving of equipment cost and operating cost. Further, the make-up hydrogen stream 66 is first used in aromatic reaction zone, where the low purity does not introduce operating problems, to provide heat for the endothermic reaction and to provide the preferred high hydrogen/hydrocarbon molar ratio. Then, the hydrogen-rich off gas is purified in a single pressure swing adsorption unit and compressed once before being consumed.

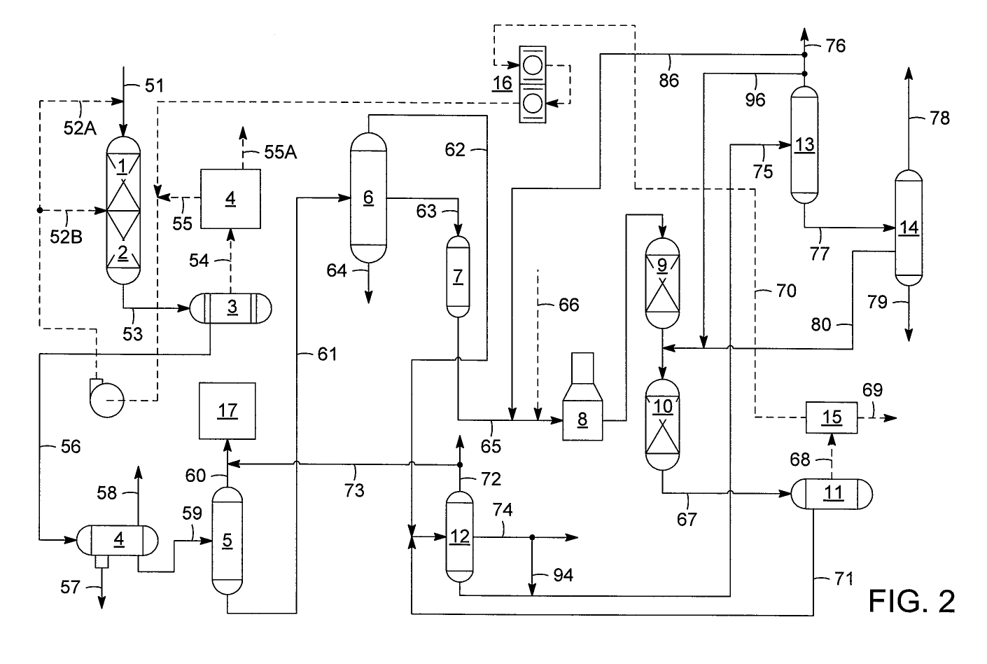

FIG. 2 illustrates an embodiment of the invention in which particular products are produced. In FIGS. 1 and 2, like reference numerals are used to illustrate like parts. Thus, for example, charge heater 8 has the same reference numeral in each drawing. This use of like numbers for the same parts identifies like parts and makes comparison of the Figures easier.

The embodiment of FIG. 2 is particularly useful for the production of aromatics and diesel from a hydrocarbon feedstock. Suitable hydrocarbon feedstocks boil in the range from about 149° C. (300° F.) to about 399° C. (750° F.) and preferably contain at least about 50 vol-% aromatic compounds. Particularly preferred feedstocks contain at least a portion of light cycle oil (LCO) which is a by-product of the fluid catalytic cracking (FCC) process. LCO is an economical and advantageous feedstock since it is undesirable as a finished product and contains significant quantities of sulfur, nitrogen and polynuclear aromatic compounds. Therefore, the present invention is able to convert a low-value LCO stream into valuable aromatic hydrocarbon compounds and diesel.

For ease of description and depiction on the FIG. 2, the term “reaction zone” may be used to refer to a plurality of reactor vessels, each having the purpose described. Similarly, reference to a reactor does not suggest that only a single reactor vessel is present. Rather, this term also refers to a plurality of reactor vessels. Each reactor shown as a single vessel in the drawing figure may indeed comprise multiple reactor vessels.

In one preferred embodiment of the present invention, the selected feedstock 51 is first introduced into a denitrification and desulfurization reaction zone 1 together with hydrogen 52A at hydrotreating reaction conditions. Preferred reaction conditions for the denitrification and desulfurization reaction and for the hydrocracking reaction are set forth above. Typical catalysts are both described herein and known to the skilled practitioner.

In accordance with this embodiment, the effluent from the hydrotreating reactor 1 is introduced to hydrocracking reactor 2, together with high-purity, high pressure hydrogen 52B.

In accordance with an embodiment illustrated in FIG. 2, the resulting effluent 53 from the hydrocracking zone 2 is introduced into a high pressure separator 3. Vapor 54 is amine treated in scrubber 4, with cleaned hydrocarbons 55 introduced to hydrotreating unit 1 and hydrocracking unit 2 as part of the hydrogen stream. Hydrogen sulfide, ammonia, and other contaminants 55A are removed from the system. Liquid 56 from high pressure separator 3 is flashed in flash drum 4, with sour water 57 removed from the system and flash gas 58 recovered.

Hydrocarbon liquids 59 are introduced into stripper 5. In an embodiment, LPG and light naphtha 60 are removed overhead and separately recovered at unit 17. A liquid stream 61 comprising C₅⁺ hydrocarbons is recovered from the bottom of stripper 5.

Liquid stream 61 is introduced to main fractionator 6 and fractionated into three main streams. Gasoline stream 62, with a boiling range of C₅ to about 195° F., is withdrawn overhead and introduced to dehexanation column 12. However, if higher benzene purity is desired, this stream is not separately recovered, but rather is further processed together with the midcut naphtha stream. The bottoms comprise diesel stream 64.

The midcut naphtha, which boils between about 185° F. and about 380° F., may be processed in desulfurizer 7 to ensure that essentially no sulfur remains therein. It is a preferred practice to operate the reforming zone 9 in a substantially sulfur-free environment. Any guard bed control means known in the art may be used to treat the naphtha feedstock 63 which is to be charged to the reforming reaction zone 9. The preferred maximum sulfur concentration in the feed stream is 2 ppm. If necessary, for example, the feedstock may be subjected to guard bed adsorption processes, guard bed catalytic processes, or combinations thereof, to maintain less than about 2 ppm sulfur in the stream.

Preferred guard bed adsorption processes may employ adsorbents such as molecular sieves, high surface area aluminas, high surface area silica-aluminas, carbon molecular sieves, crystalline aluminosilicates, activated carbons, and high surface area metallic containing compositions, such as nickel or copper and the like. Guard beds may be loaded in separate vessels in the reforming zone 9 or the hydrocracking zone 2, or guard beds may be loaded inside the catalyst vessel or vessels themselves. Guard beds may also be loaded in conjunction with the transalkylation zone as needed to deal with any contaminants such as sulfur or chloride that may arise from specific streams passing over the transalkylation catalyst. Resultant desulfurized naphtha 65 then may be combined with make-up hydrogen 66 and overhead stream 76 from toluene fractionator column 13 and introduced to charge heater 8.

In this embodiment, make-up hydrogen stream 66 typically is from a low-purity supply, such as from a hydrogen plant or from a naphtha reformer. The endothermic dehydrogenation step carried out in dehydrogenator 9 requires heat, which is carried through the reactor by the relatively large gas flow. Thus, no inter-reactor heating is required. Typical operating conditions and catalysts are described above.

In embodiments of the invention, it is possible to use only a dehydrogenator, only a transalkylation reactor, or both. The embodiment illustrated in FIG. 2 shows both dehydrogenation reactor or zone 9 and transalkylation reactor or zone 10. However, both processes are not required in all embodiments of the invention.

In the transalkylation zone 10, the reformate feedstock is preferably transalkylated in the vapor phase and in the presence of hydrogen. Toluene and aromatic hydrocarbons of carbon number nine and above may also be fed directly to the transalkylation zone part and thus bypass the reforming zone part. Such a stream may be obtained from xylenes column 14 as stream 80, which is the cut between xylenes overhead and A₁₁⁺ bottoms. If present, free hydrogen present in an amount of from about 0.1 moles per mole of alkylaromatics up to 10 moles per mole of alkylaromatic. This ratio of hydrogen to alkylaromatic is also referred to as hydrogen to hydrocarbon ratio. The transalkylation reaction preferably yields a product 67 having an increased xylene content and also comprises toluene.

The feed to a transalkylation reaction zone 10 usually first is cooled by quench with incoming streams 96 and/or 80, and/or by indirect heat exchange against the effluent of the reaction zone and then is further cooled to reaction temperature by exchange with a cooler stream or boiler feed water for steam production. The use of a single reaction vessel having a fixed cylindrical bed of transalkylation catalyst is preferred, but other reaction configurations utilizing moving beds of catalyst or radial-flow reactors may be employed if desired. Passage of the combined feed through the reaction zone 10 effects the production of an effluent stream 67 comprising unconverted feed and product hydrocarbons. This effluent is normally cooled by indirect heat exchange against the stream entering the reaction zone and then further cooled through the use of air or cooling water.

To effect a transalkylation reaction, the present invention incorporates a transalkylation catalyst in at least one zone, but no limitation is intended in regard to a specific catalyst. Conditions employed in the transalkylation zone 10 normally include a temperature of from about 200° C. (392° F.) to about 540° C. (1004° F.). The transalkylation zone 10 is operated at moderately elevated pressures broadly ranging from about 100 kPa (14.7 psig) to about 6 MPa (870 psig). The transalkylation reaction can be effected over a wide range of space velocities. Liquid hourly space velocity generally ranges from about 0.1 to about 20 hr⁻¹.

Any suitable transalkylation catalyst may be utilized in the transalkylation zone. Preferred transalkylation catalysts contain a solid-acid material combined with an optional metal component. Suitable solid-acid materials include all forms and types of mordenite, mazzite (omega zeolite), beta zeolite, ZSM-11, ZSM-12, ZSM-22, ZSM-23, MFI type zeolite, NES type zeolite, EU-1, MAPO-36, MAPSO-31, SAPO-5, SAPO-11, SAPO-41, and silica-alumina or ion exchanged versions of such solid-acids. For example, in U.S. Pat. No. 3,849,340 a catalytic composite is described comprising a mordenite component having an SiO₂/Al₂O₃ mole ratio of at least 40:1 prepared by acid extracting Al₂O₃ from mordenite prepared with an initial SiO₂/Al₂O₃ mole ratio of less than 30:1 and a metal component selected from copper, silver and zirconium. Refractory inorganic oxides, combined with the above-mentioned and other known catalytic materials, have been found useful in transalkylation operations. For instance, silica-alumina is described in U.S. Pat. No. 5,763,720. Crystalline aluminosilicates have also been employed in the art as transalkylation catalysts. ZSM-12 is more particularly described in U.S. Pat. No. 3,832,449. Zeolite beta is more particularly described in Re. 28,341 (of original U.S. Pat. No. 3,308,069). A favored form of zeolite beta is described in U.S. Pat. No. 5,723,710, which is incorporated herein by reference. The preparation of MFI topology zeolite is also well known in the art. In one method, the zeolite is prepared by crystallizing a mixture containing an alumina source, a silica source, an alkali metal source, water and an alkyl ammonium compound or its precursor. Further descriptions are in U.S. Pat. No. 4,159,282, U.S. Pat. No. 4,163,018, and U.S. Pat. No. 4,278,565. The synthesis of the Zeolite Omega is described in U.S. Pat. No. 4,241,036. ZSM intermediate pore size zeolites useful in this invention include ZSM-5 (U.S. Pat. No. 3,702,886); ZSM-11 (U.S. Pat. No. 3,709,979); ZSM-12 (U.S. Pat. No. 3,832,449); ZSM-22 (U.S. Pat. No. 4,556,477); ZSM-23 (U.S. Pat. No. 4,076,842). European Patent EP 0378916 B1 describes NES type zeolite and a method for preparing NU-87. The EUO structural-type EU-1 zeolite is described in U.S. Pat. No. 4,537,754. MAPO-36 is described in U.S. Pat. No. 4,567,029. MAPSO-31 is described in U.S. Pat. No. 5,296,208 and typical SAPO compositions are described in U.S. Pat. No. 4,440,871 including SAPO-5, SAPO-11 and SAPO-41.

A refractory binder or matrix is optionally utilized to facilitate fabrication of the transalkylation catalyst, provide strength and reduce fabrication costs. The binder should be uniform in composition and relatively refractory to the conditions used in the process. Suitable binders include inorganic oxides such as one or more of alumina, magnesia, zirconia, chromia, titania, boria, thoria, phosphate, zinc oxide and silica. Alumina is a preferred binder.

The transalkylation catalyst also may contain an optional metal component. One preferred metal component is a Group VIII (IUPAC₈₋₁₀) metal that includes nickel, iron, cobalt, and platinum-group metal. Of the platinum group, i.e., platinum, palladium, rhodium, ruthenium, osmium and iridium, platinum is especially preferred. Another preferred metal component is rhenium and it will be used for the general description that follows. This metal component may exist within the final catalytic composite as a compound such as an oxide, sulfide, halide, or oxyhalide, in chemical combination with one or more of the other ingredients of the composite. The rhenium metal component may be incorporated in the catalyst in any suitable manner, such as coprecipitation, ion-exchange, co-mulling or impregnation. The preferred method of preparing the catalyst involves the utilization of a soluble, decomposable compound of rhenium metal to impregnate the carrier material in a relatively uniform manner. Typical rhenium compounds which may be employed include ammonium perrhenate, sodium perrhenate, potassium perrhenate, potassium rhenium oxychloride, potassium hexachlororhenate (IV), rhenium chloride, rhenium heptoxide, perrhenic acid, and the like compounds. Preferably, the compound is ammonium perrhenate or perrhenic acid because no extra steps may be needed to remove any co-contaminant species.

The transalkylation catalyst may optionally contain additional metal components along with those metal components discussed above or include additional metal components instead of those metal components in their entirety. Additional metal components of the catalyst include, for example, tin, germanium, lead, and indium and mixtures thereof. Catalytically effective amounts of such additional metal components may be incorporated into the catalyst by any means known in the art.

The resulting effluent 67 from the transalkylation zone 10 is introduced into a vapor-liquid separator 11 to provide a hydrogen-rich gaseous stream 68 which is subjected to purification in pressure swing adsorber 15. Tail gas 69 is separately recovered.

In pressure swing adsorber 15, the purity of the hydrogen product 70 is increased to at least about 99.5 wt percent, more typically about 99.8 wt percent, and even more typically 99.9 wt percent. Essentially pure hydrogen stream 70 is compressed in one or more stages of compressor 16 and fed, together with stream 55 from amine scrubber 4, at high pressure to hydrotreating zone 1 as stream 52A and hydrocracking zone 2 as stream 52B, where it is essentially completely consumed.

Thus, this once-through processing of hydrogen in accordance with embodiments of the invention produces a significant saving of equipment cost and operating cost. Further, the make-up hydrogen stream 66 is first used in dehydrogenator 9 and transalkylation reactor 10, where the low purity does not introduce operating problems, to provide heat for the endothermic reaction and to provide the preferred high hydrogen/hydrocarbon molar ratio. Then, the hydrogen-rich off gas is purified in a single pressure swing adsorption unit and compressed once before being consumed.

The naphtha boiling range C₅⁺ liquid aromatic-containing hydrocarbon 71 from the vapor-liquid separator 11 is fractionated in dehexanation column 12. In an embodiment, gasoline stream 62 is combined with the feed to the dehexanation column 12 to remove hydrocarbons boiling at a temperature lower than benzene stream 72. Part of this stream can, in an embodiment, be sent to LPG and light naphtha recovery at unit 17 as stream 73. The remainder is removed from the system.

In one embodiment, benzene cut 94 is combined with bottoms cut 75 and further fractionated in toluene column 13. Overhead stream 96 from toluene column 13 then is returned to the transalkylation reaction 10. This embodiment maximizes xylenes production. In another embodiment, to produce more benzene and toluene at the expense of xylene production, streams 74 and 76 may be directed to product tanks. For example, if the operator wishes to produce additional high octane gasoline, the benzene/toluene net product flow rate would be increased and the overall xylene production would thereby decrease. This readily available feature affords a very flexible way to produce different product slates.

Another preferred embodiment also is illustrated in FIG. 2. In this embodiment, benzene 74 is removed from the system, and bottoms stream 75 is provided to toluene column 13. Overhead toluene stream 76 is recycled to be combined with light naphtha 65.

In all embodiments, bottoms 77 from toluene column 13 are directed to transalkylation/cracking column 14 for additional fractionation. In embodiments of the invention, three streams are withdrawn from transalkylation/cracking column 14. The overhead stream 78 comprises the xylenes product. Bottoms 79 comprise aromatic compounds having at least about 10 carbon atoms. Aromatic hydrocarbons having about 9 or about 10 carbon atoms are withdrawn as stream 80 and, in the illustrated embodiment, returned directly to the feed to the transalkylation/cracking reactor 10.

As can be seen, embodiments of the invention provide a multi-zone process comprising a hydrocracking zone and an aromatics zone for production of diesel and aromatics wherein the need for a recycle gas compressor in the aromatics zone is reduced or eliminated and the hydrogen circulation path is made ‘once through,’ i.e., no significant recycle is required. As is typical for such a process, an impure hydrogen-containing stream serves as a hydrogen source for the process. However, in embodiments of the invention, the impure stream is introduced into the charge heater for the aromatics section and serves to reduce the need to compensate for the endothermic heat of reaction in the dehydrogenation reaction and to essentially eliminate the need for a hydrogen-containing recycle around the dehydrogenator and the transalkylation reactor in the aromatics zone. Embodiments of the invention also provide a ‘once-through’ flow path for the hydrogen make-up and reduce the number of pressure swing adsorption units for hydrogen purification to one. In accordance with embodiments of the invention, the hydrogen needs of the processes are better balanced, because the hydrogen is essentially consumed in the hydrotreating and hydrocracking reaction steps.

While the invention has been described with respect to specific examples including presently preferred modes of carrying out the invention, those skilled in the art will appreciate that there are numerous variations and permutations of the above described systems and techniques that fall within the spirit and scope of the invention as set forth in the appended claims.

1. A method for integrating the hydrogen flow in a product-producing reaction system having at least one first reaction unit that consumes a large quantity of hydrogen at high purity and at least one second reaction unit that does not consume a large quantity of hydrogen, but which requires the presence of hydrogen and can tolerate the presence of hydrogen at low purity, said method comprising:
(a) reacting a first reactant and an essentially pure hydrogen stream in the at least one first reaction unit to produce a first effluent stream;
(b) fractionating the first effluent stream to recover at least one first product stream;
(c) introducing a low-purity hydrogen-containing stream to the at least one first product stream from step (b) to form a combined stream;
(d) reacting the combined stream to form a second product stream comprising hydrogen, volatile compounds, and a product;
(e) separating the hydrogen and volatile components from the product to form a gaseous stream and a product-containing stream;
(f) purifying the gaseous stream to provide an essentially pure hydrogen-containing stream;
(g) increasing the pressure on the essentially pure hydrogen-containing stream and introducing it to the at least one first reaction unit of step (a); and
(h) recovering the product.

2. A method for balancing hydrogen needs in a petroleum refinery, said method comprising
feeding an impure hydrogen stream to an aromatics zone,
reacting the impure hydrogen stream with other hydrocarbons in the aromatics zone to produce an effluent,
separating a hydrogen-rich stream from the effluent,
purifying the hydrogen-rich stream to yield an essentially pure hydrogen stream, and
compressing the essentially pure hydrogen stream and feeding the essentially pure hydrogen stream to high-pressure hydrotreating and hydrocracking reactors.

3. The method of claim 2, wherein the aromatics zone comprises at least a dehydrogenation reactor and the other hydrocarbons comprise a light naphtha stream.

4. The method of claim 1, wherein the conversion in step (d) is enhanced by the inclusion of contaminants in the low-purity hydrogen-containing stream.

5. A method for efficiently using hydrogen in aromatics production from heavy aromatic oil, said method comprising:
(a) contacting a hydrocarbonaceous stream comprising C₉⁺ hydrocarbons and an essentially pure hydrogen stream serially with a hydrotreating catalyst under hydrotreating conditions and a hydrocracking catalyst under hydrocracking conditions in a hydrocracking zone and, respectively, to produce a hydrocracking zone effluent comprising xylenes;
(b) fractionating the hydrocracking zone effluent in a fractionation zone to separately recover a first stream of hydrocarbons comprising C₄ and lighter hydrocarbons, a second stream of hydrocarbons comprising hydrocarbons boiling below about 195° F., a third stream comprising hydrocarbons boiling between about 180° F. and about 380° F., and a fourth stream comprising diesel;
(c) introducing a low purity hydrogen-containing stream to the third stream from step (b) to form a combined stream and heating the combined stream to form a heated combination;
(d) contacting the heated combination with a dehydrogenation catalyst to form a dehydrogenated stream;
(e) separating the hydrogen and volatile components from the aromatics to form a gaseous stream and an aromatic-containing stream;
(f) subjecting the gaseous stream to pressure swing adsorption to provide an essentially pure hydrogen-containing stream;
(g) increasing the pressure on the essentially pure hydrogen-containing stream and introducing it to the hydrocracking zone of step (a); and
(h) recovering the liquid hydrocarbon products in the aromatic-containing stream.

6. The method of claim 5, further comprising contacting the dehydrogenated stream of step (d) with transalkylation catalyst to form an effluent comprising hydrogen, volatile compounds, and aromatics.

7. The method of claim 5, further comprising fractionating the aromatics-containing stream of step (h) to form a benzene fraction, a toluene fraction, and a xylenes fraction.

8. The method of claim 6, further comprising fractionating the aromatics-containing stream of step (h) to form a benzene fraction, a toluene fraction, and a xylenes fraction.

9. The method of claim 6, further comprising recycling the toluene fraction to the transalkylation reactor.

10. The method of claim 8, further comprising recycling the toluene fraction to the transalkylation reactor.

11. The method of claim 7, further comprising fractionating the xylenes fraction to form a stream containing C₉-C₁₀ alkylaromatic compounds, mixing that stream with the dehydrogenated stream of step (d), and contacting the mixture with the transalkylation catalyst.

12. The method of claim 8, further comprising fractionating the xylenes fraction to form a stream containing C₉-C₁₀ alkylaromatic compounds, mixing that stream with the dehydrogenated stream of step (d), and contacting the mixture with the transalkylation catalyst.

13. The method of claim 9, further comprising fractionating the xylenes fraction to form a stream containing C₉-C₁₀ alkylaromatic compounds, mixing that stream with the dehydrogenated stream of step (d), and contacting the mixture with the transalkylation catalyst.

14. The method of claim 10, further comprising fractionating the xylenes fraction to form a stream containing C₉-C₁₀ alkylaromatic compounds, mixing that stream with the dehydrogenated stream of step (d), and contacting the mixture with the transalkylation catalyst.

15. A method for efficiently using hydrogen in aromatics production from heavy aromatic oil, said method comprising:
(a) contacting a hydrocarbonaceous stream comprising C₉⁺ hydrocarbons and an essentially pure hydrogen stream serially with a hydrotreating catalyst under hydrotreating conditions and a hydrocracking catalyst under hydrocracking conditions in a hydrocracking zone and, respectively, to produce a hydrocracking zone effluent comprising xylenes;
(b) fractionating the hydrocracking zone effluent in a fractionation zone to separately recover a first stream of hydrocarbons comprising C₄ and lighter hydrocarbons, a second stream of hydrocarbons comprising hydrocarbons boiling below about 195° F., a third stream comprising hydrocarbons boiling between about 180° F. and about 380° F., and a fourth stream comprising diesel;
(c) introducing a low purity hydrogen-containing stream to the third stream from step (b) to form a combined stream and heating the combined stream to form a heated combination;
(d) contacting the heated combination with a transalkylation catalyst to form an effluent comprising hydrogen, volatile compounds, and aromatics;
(e) separating the hydrogen and volatile components from the aromatics to form a gaseous stream and an aromatic-containing stream;
(f) subjecting the gaseous stream to pressure swing adsorption to provide an essentially pure hydrogen-containing stream;
(g) increasing the pressure on the essentially pure hydrogen-containing stream and introducing it to the hydrocracking zone of step (a); and
(h) recovering the liquid hydrocarbon products in the aromatic-containing stream.

16. The method of claim 15, further comprising fractionating the aromatics-containing stream of step (h) to form a benzene fraction, a toluene fraction, and a xylenes fraction.

17. The method of claim 15, further comprising recycling the toluene fraction to the transalkylation reactor.

18. The method of claim 16, further comprising recycling the toluene fraction to the transalkylation reactor.

19. The method of claim 15, further comprising fractionating the xylenes fraction to form a stream containing C₉-C₁₀ alkylaromatic compounds, mixing that stream with the dehydrogenated stream of step (d), and contacting the mixture with the transalkylation catalyst.

20. The method of claim 16, further comprising fractionating the xylenes fraction to form a stream containing C₉-C₁₀ alkylaromatic compounds, mixing that stream with the dehydrogenated stream of step (d), and contacting the mixture with the transalkylation catalyst.
